(19) United States
(12) Patent Application Publication
(10) Pub. No.: US 2005/0220865 A1
(43) Pub. Date: Oct. 6, 2005

(54) Compressed composition comprising magnesium salt

(76) Inventors: John J. Koleng, Austin, TX (US); Michael M. Crowley, Austin, TX (US)

Correspondence Address: INNOVAR, LLC, P O BOX 250647, PLANO, TX, 75025, US

(21) Appl. No.: 10/816,771

(22) Filed: Apr. 2, 2004

(51) Int. Cl. A61K 9/48; A61K 9/20; A61K 33/08

(52) U.S. Cl. 424/451; 424/464; 424/692

(57) ABSTRACT

An oral solid compress composition comprising a magnesium salt is provided. The composition provides a rapid dissolution of the magnesium salt, wherein not less than 75% of the magnesium salt dissolves within 45 minutes after placement in hydrochloric acid (0.1 N, 900 mL) as per USP Method <711>. In a particular embodiment, the magnesium salt is an inorganic salt such as MgO, Mg(OH)₂, MgCl₂, and others. The composition can be prepared by dry granulation, direct compression or another suitable process. The composition provides a substantially stable dissolution profile for the magnesium salt so that the dissolution profile changes only minimally even after an extended period of storage under pharmaceutically acceptable conditions when packaged in a sealed container-enclosure system. The solid composition may also exclude a cellulose-based composition. The compressed composition can be prepared and stored under anhydrous conditions.

### FIELD OF THE INVENTION

The present invention relates to a rapidly disintegrating compressed oral solid composition that provides a rapid dissolution of magnesium salt. The invention also relates to solid oral dosage forms containing the composition. The invention also provides methods for preparation and use thereof.

### BACKGROUND OF THE INVENTION

Magnesium is an essential mineral in human nutrition with a wide range of biological functions. The total body magnesium content of an adult is about 25 grams. About 50%-60% exists in bone. Magnesium is involved in over 300 metabolic reactions. It is necessary for major biological processes including the production of cellular energy and the synthesis of nucleic acids and proteins. It is also important for the electrical stability of cells, the maintenance of membrane integrity, muscle contraction, nerve conduction and the regulation of vascular tone, among other things.

Symptoms and signs of magnesium deficiency include anorexia, nausea and vomiting, diarrhea, generalized muscle spasticity, paresthesias, confusion, tremor, focal and generalized seizures, confusion, loss of coordination, cardiac arrhythmias, laboratory abnormalities, such as hypokalemia and hypocalcemia, muscle cramps, hypertension and coronary and cerebral vasospasms. Magnesium deficiency may be found in diabetes mellitus, malabsorption syndromes, alcoholism and hyperthyroidism, among other disorders. Use of certain drugs may also lead to magnesium deficiency. These drugs include thiazide diuretics (when used for long periods of time), loop diuretics, cisplatin, amphotericin, pentamidine (when used intravenously), aminoglycosides and cyclosporine. Magnesium deficiency itself is an important cause of hypokalemia.

Given the above, a significant effort has been made to develop and commercialize a spectrum of oral dosage forms containing magnesium salt. Commercially available capsules and tablets comprising therapeutic amounts of a magnesium salt are widely known. Tablets are available in rapid release form under the following trademarks. DOCTORS' BEST™ high absorption magnesium glycinate contains magnesium bis-glycinate, microcrystalline cellulose, croscarmellose sodium, magnesium stearate, stearic acid. RAINBOW LIGH™ magnesium citrate contains magnesium citrate, potassium citrate, pyridoxal-5′-phosphate, microcrystalline cellulose, stearic acid (vegetable), silica, magnesium stearate. JARROW FORMULAS™ magnesium citrate QUIK-SOLV™ tablets contains magnesium citrate, potassium chloride, cellulose, stearic acid, modified cellulose gum, silicon dioxide, and magnesium stearate. SOURCE NATURALS™ magnesium amino acid chelate contains magnesium chelate, calcium, sorbitol, dibasic calcium phosphate, stearic acid, modified cellulose gum, colloidal silicon dioxide, vegetable fiber and magnesium stearate. COUNTRY LIFE™ calcium magnesium complex contains calcium hydroxyapatite, magnesium oxide, magnesium citrate, magnesium taurinate, magnesium aspartate, magnesium α-keto-glutarate, cellulose, stearic acid, silica, cellulose and a glycerin coating. MAOX™, SOLGAR™ chelated magnesium contains magnesium glycinate amino acid chelate, microcrystalline cellulose, dicalcium phosphate, vegetable cellulose, vegetable stearic acid. MALDROXAL™ contains aluminum/magnesium hydroxide. RIPPLE CREEK® magnesium oxide tablet contains magnesium oxide. HILLESTAD® magnesium oxide contains magnesium oxide. SeaPlex™ magnesium tablet contains Spirulina, Cholera, Irish Moss, Kelp, Bladderwack, cellulose, potato starch, magnesium stearate, and silicon dioxide. NUTRITION DYNAMICS INC. magnesium supplement tablets contain MgO, magnesium glycinate, magnesium aspartate, magnesium amino acid chelate, or magnesium taurinate or a combination of MgO, magnesium carbonate and magnesium sulfate. VITAMIN WORLD® NATURALLY INSPIRED™ magnesium contains magnesium oxide, cellulose (plant origin), vegetable stearic acid, vegetable magnesium stearate, silica. VITAMIN WORLD® NATURALLY INSPIRED™ magnesium citrate contains cellulose (plant origin), vegetable stearic acid, starch, croscarmellose, silica, vegetable magnesium stearate, cellulose coating. PURITAN'S PRIDE® INSPIRED BY NATURE™ magnesium contains cellulose (plant origin), vegetable stearic acid, vegetable magnesium stearate, silica. VALU-RITE® magnesium contains magnesium oxide, calcium carbonate, microcrystalline cellulose, modified cellulose gum, stearic acid, citric acid, magnesium stearate, talc. SWISS® NATURAL SOURCES® magnesium oxide contains magnesium oxide among other things. CYPRESS PHARMACEUTICAL INC. magnesium oxide contains magnesium oxide, dicalcium phosphate, microcrystalline cellulose, stearic acid, magnesium stearate and silica. GENESIS PRODUCTS INC. magnesium oxide contains magnesium oxide, microcrystalline cellulose, corn starch, magnesium stearate and optionally talc. LEINER HEALTH PRODUCTS YOURLIFE™ magnesium oxide contains magnesium oxide, cellulose (unspecified type), talc, sodium starch glycolate, starch, silicon dioxide, croscarmellose sodium, polyethylene glycol, kelp, magnesium stearate.

Magnesium supplements are available in capsule form under the following trademarks. TWIN LABS™ magnesium caps contain magnesium oxide and magnesium aspartate in a hard gelatin capsule. NUTRITION DYNAMICS INC. magnesium supplements capsules contain magnesium glycinate, carnitine, and taurine; or magnesium aspartate or magnesium citrate. LIFE EXTENSION® magnesium contains magnesium oxide, magnesium chloride, magnesium succinate, magnesium amino acid chelate, and rice flour, magnesium stearate, gelatin and water. SELF HEALTH® magnesium contains magnesium oxide and gelatin. DOUGLAS LABORATORIES® magnesium contains magnesium oxide among other things.

Others magnesium salt supplements such as ALMORA™ (Forest Pharmaceuticals), CHLOROMAG™, CITROMA™, MAGONATE™ (Fleming and Company), MAGTRATE™ (Mission Pharmacal), MGP™, PHILLIPS'® Chewable Tablets, CITRO-MAG™, MAGLUCATE™, URO-MAG (Blaine Pharmaceuticals), MAG-OX 400 (Blaine Pharmaceuticals), MAGNACAPS (The Key Company), M2 magnesium (Miller Pharmacal), MAGIMIN-FORTE (The Key Company), ELITE magnesium (Miller Pharmacal), and MA-G (Cypress Pharmaceuticals) are also available. Geist Pharmaceuticals, LLC distributes MAGINEX™. The Geist product provides 122 mg of magnesium per tablet from 1230 mg of an L-Aspartate hydrochloride magnesium salt.

A number of scientific literature and patent references disclose the administration of magnesium salts (magnesium oxide, magnesium hydroxide, magnesium citrate, magnesium gluconate, magnesium chloride, magnesium aspartate, magnesium glycinate and others) as antacids. Other references disclose the use of magnesium oxide or magnesium hydroxide as a stabilizing agent in tablet formulations.

U.S. Pregrant Publication No. 2003017536 to Luzzatti discloses oral tablet dosage forms containing an antacid (such as a magnesium salt) and a local anesthetic. The tablet can be a compressed tablet, chewable tablet, a quick dissolve tablet, or an effervescent tablet, among other things.

Japanese Patent Application No. 2002255802 to Kitayama et al. discloses rapidly acting antipyretic analgesic oral dosage forms for the treatment of cold. The preparations comprise ibuprofen and magnesia at the wt. ratio of 100 to 50-150 and may further comprise allylisopropylacetylurea and caffeine. An exemplary tablet comprises ibuprofen (75 mg), MgO (83.3 mg), allylisopropylacetylurea (30 mg), caffeine (40 mg), and miscellaneous excipients (75 mg).

Yong et al. (Drug Development and Industrial Pharmacy (2001), 27(5), 447-455) disclose an omeprazole-containing buccal adhesive tablet with excellent bioadhesive strength and good drug stability in human saliva. An exemplary tablet comprises omeprazole/sodium alginate/HPMC/magnesium oxide/croscarmellose sodium (20/24/6/50/10 mg). This could be attached to the cheek without disintegration.

U.S. Pat. No. 6,024,987 to Jettka et al. discloses “a pharmaceutical, orally applicable solid composition wherein the solid composition contains at least one antacid active ingredient, . . . at least one disintegrant selected from the group consisting of starch, a starch derivative, cellulose, a cellulose derivative, alginic acid, an alginic acid derivative, casein, a casein derivative, an insoluble polyvinylpyrrolidone, and mixtures thereof, at least one usual pharmaceutical additional ingredient, and at least one ingredient accelerating the decomposition of said composition in the mouth or in a liquid, wherein said ingredient is selected from the group consisting of glycine, proline, hydroxy proline, lysine, and the salts and derivatives thereof, wherein said composition contains said ingredient in such a concentration that the composition decomposes in the mouth or in a liquid within one to thirty seconds.” The antacid is selected from the group consisting of “aluminum-hydroxide, magnesium-hydroxide, magnesium-trisilicate, magnesium-carbonate, magnesium-phosphate, calcium-carbonate, calcium-phosphate, sodium-citrate, magnesium-dioxide.”

European Patent No. EP 761227 to Shiozawa discloses a solid oral dosage form comprising two different types of acid neutralizing compounds, a low neutralizing antacid and a high neutralizing antacid. The dosage form is a combination rapid and slow release tablet. The high neutralizing antacid (such as magnesium hydroxide, magnesium oxide, magnesium carbonate, magnesium silicate) is released slowly over a period of about three hours and the low neutralizing antacid (such as aluminum-magnesium composite compound antacids such as magnesium aluminate, dimagnesium silicate aluminate, magnesium metasilicate aluminate, magnesium bismuth silicate aluminate and synthetic hydrotalcite; and aluminum compound antacids such as dried aluminum hydroxide gel and aluminum silicate) is released rapidly after administration. The dosage form can be a tablet, chewable tablet, granule, powder, fine granule, pill or capsule.

Japanese Patent No. 5229936 to Nishikawa discloses an oral granular mixture that disintegrates rapidly after administration and reportedly has “good storage stability.” The granular mixture comprises two different types of granules: a first group of granules containing acetaminophen and a second group of granules containing a water-soluble polymer dispersed within an antacid. An exemplary mixture comprises granules (1 g) made from MgO (500 g), MgCO3 (1000 g), corn starch (200 g), hydroxypropyl methylcellulose (100 g), and sucrose fatty acid ester (50 g), and granules (1 g) made from acetaminophen (1000 g), starch (200 g), and corn starch (100 g). The granules reportedly showed good stability when stored at 50° and 75% relative humidity for 1 month.

U.S. Pat. No. 5,035,898 to Chang et al. discloses a solid oral dosage form that provides a controlled release of potassium chloride and an immediate release of magnesium salt, esp. magnesium oxide.

Japanese Patent No. 2906528 to Taisho Pharmaceutical Co. discloses a solid oral dosage form that reportedly provides a rapid onset of analgesic activity after administration. The tablet comprises an antacid (such as sodium hydrogen carbonate, calcium hydrogen phosphate, aluminum magnesium silicate, MgO and synthetic hydrotalcitee) and a piroxicam-type of analgesic. An exemplary dosage form comprises 2 mg of chlortenoxicam, 20 mg of sodium hydrogen carbonate, 50 mg of calcium hydrogen phosphate anhydrous GS, 40 mg of microcrystalline cellulose, 2.5 mg of aelogil, 25 mg of substituted hydroxypropyl cellulose and 0.5 g of calcium stearate.

U.S. Pat. No. 4,115,553 to Margres et al. discloses a chewable antacid tablet formulation comprising 10-90% of a co-dried combination of a hydrous gelatinous Al hydroxide material with an alcohol and an excipient. The Al compound is basic Al bicarbonate-carbonate optionally in combination with basic Mg carbonate, Mg hydroxide and/or Mg trisilicate.

U.S. Pat. No. 3,257,275 to Weisberg discloses a rapidly acting oral tablet comprising an antacid. An exemplary composition comprises chitosan (1000 parts), MgO (50 parts), lactose (150 parts), and Mg stearate (10 parts).

U.S. Pat. No. 4,599,152 to Ashmead discloses amino acid chelates of magnesium, among other metals.

U.S. Pat. No. 6,521,256, U.S. Pat. No. 6,380,234, U.S. Pat. No. 6,296,875, U.S. Pat. No. 6,123,962, U.S. Pat. No. 6,017,560, U.S. Pat. No. 5,879,702, U.S. Pat. No. 5,639,478, U.S. Pat. No. 5,433,959, U.S. Pat. No. 5,093,132 and U.S. Pat. No. 5,045,321 to Makino et al. disclose a stabilized benzimidazole composition comprising a benzimidazole compound and a magnesium salt.

U.S. Pat. No. 6,569,477 to Lederman et al. discloses reportedly highly soluble mineral supplements containing calcium and magnesium. The reconstitutable powder formulations reportedly possess increased solubility for the calcium salt. The reconstitutable powder is made by solubilizing the metal salts completely in an acidic solution and drying the solution. Lederman et al. suggest that the material can be used in tablets and several examples of the powder include MgO.

U.S. Pregrant Publication No. 20030129228 to Kay discloses a dual active agent dosage form that provides a controlled release of a magnesium salt. The first active agent reduces the rate of absorption of the magnesium salt.

U.S. Pat. No. 6,589,507 to Bauer et al. discloses an effervescent antacid tablet, preferably chewable, comprising “(i) an antacid, (ii) an effervescent mixture that releases CO₂, (iii) a polymeric surfactant as foam-forming agent or a mixture of such surfactants, (iv) a swellable and gel-forming polymer or a mixture of such polymers, and (v) optionally conventional auxiliary substances.” The antacid can be “magnesium hydroxide, magnesium oxide, magnesium carbonate, magnesium silicate, aluminum hydroxide, aluminum phosphate and magnesium aluminum silicate or mixtures thereof.”

U.S. Pregrant Publication No. 20030068373 to Luber discloses a rapid release compressed tablet made from a hydrophobic wax matrix. The tablet comprises at least 60% wt. of an active ingredient and powdered wax having a melting point greater than about 90° C. The active ingredient can be “acetaminophen, ibuprofen, calcium carbonate, magnesium hydroxide, magnesium carbonate, magnesium oxide, aluminum hydroxide, mixtures thereof, and pharmaceutically acceptable salts thereof.” A disintegrant may also be added and the tablet can be coated. Luber states that the tablet meets the USP specification for immediate release tablets. Polyalkylene glycols and polyethylene oxides are listed as suitable powdered waxes. The tablet can have a hardness in the range of about 4 to 20 kp/cm².

U.S. Pat. No. 6,417,196 to Daniel et al. discloses a stabilized dosage form of quinapril and magnesium oxide.

U.S. Pat. No. 5,320,852 to Moest et al. discloses antacid biconvex tablets whose height and diameter are approximately equal and are from 1.5 to 4 mm. The antacid can be “magnesium hydroxide, magnesium oxide, magnesium carbonate, magnesium silicate, aluminum hydroxide, aluminum phosphate, magnesium aluminum silicate, and mixtures thereof.” Disintegration times were about 8-9 min. for exemplary tablets. The composition of exemplary tablets optionally includes microcrystalline cellulose.

U.S. Pat. No. 5,318,858 to Cohen et al. discloses an antacid composition “comprising, in association with a physiologically acceptable excipient, a pharmaceutically effective amount of a mixture of (a) an Al-containing material which normally adheres to the gastrointestinal mucous membrane, as a first pharmaceutically active component, and (b) a Mg-containing material which normally does not adhere to the gastrointestinal mucous membrane, as a second pharmaceutically active component, wherein the weight ratio of Al in said Al-containing material to Mg in said Mg-containing material is 5.1:1 to 7.0:1.” The magnesium containing material can be MgO or Mg(OH)₂. Cohen et al. do not disclose the use of cellulose. There is no specific mention of the rate of disintegration or dissolution.

U.S. Pat. No. 4,104,370 to Bloch discloses an antacid tablet comprising magnesium and potassium for treating depletion of those metals. The tablet, however, provides a controlled release of the two metals.

European Patent No. EP 1004311 to Inoue et al. discloses a laxative composition that can be a tablet. The composition comprises an “active MgO which has a BET value (surface area in terms of m²/g) of at least 21, preferably 21-50, more preferably 30-40 and is excellent in acid reactivity, and may preferably be incorporated with lactic acid bacteria, a mixture of sporolactobacteria and yeast extracts and/or oligosaccharides.”

U.S. Pat. No. 4,446,135 to Fountaine discloses a chewable antacid tablet comprising “45 to 50 weight percent of calcium carbonate and from 8 to 11 weight percent of magnesium hydroxide, as the effective antacid ingredients, along with from 30 to 40 weight percent of sucrose and from 8 to 11 weight percent of mannitol.”

European Patent No. EP 578732 corresponding to PCT International Publication No. WO 92/17161 to Upson et al. discloses a chewable antacid tablet comprising a pregranulated antacid composition and granulated mannitol. The tablet comprises about 25% to 75% of the granulated mannitol. The antacid can be “magnesium aluminate, magnesium alumino silicates, magnesium carbonate, magnesium glycinate, magnesium hydroxide, magnesium oxide, magnesium trisilicate” among other things. The pregranulated composition comprises more than about 50% of antacid agent by weight of the pre-granulate, and less than about 50% of a granulating agent. The tablet reportedly has a quick disintegration time.

A number of the above-mentioned tablets contain microcrystalline cellulose, while others do not. Generally, the tablets and caplets are intended for rapid disintegration and release of magnesium salt. Some embodiments of the above-mentioned commercially available tablets, such as those of Leiner Health Products, meet the USP <711> guidelines for dissolution of magnesium oxide tablets. The USP27/NF22 monograph for magnesium oxide tablets, USP specifies that at least 75% of the labeled amount of MgO must be dissolved in 45 minutes under the prescribed test conditions. The present inventors, however, have discovered that the commercially available tablets formulations fail to meet the USP <711> monograph guidelines for MgO tablets after extended storage of at least 2 or more months at 40° C. and 75% relative humidity. The evaluation of products stored at 40° C. and 75% relative humidity is used to estimate the expiry dating of products typically handled at 25° C. and 60% relative humidity. Failure to initially meet USP specifications will result in lower absorption of the magnesium salt since the greatest percentage of magnesium salt absorption occurs in the acidic portion of the upper GI tract. Accordingly, commercially available dosage forms that initially meet the USP specifications will fail to provide sufficient amounts of magnesium to a subject if the dosage forms are stored for an extended period of time.

Therefore, even with all of the above-mentioned formulations available, there remains a need for an improved oral solid composition that meets the magnesium oxide tablet USP <711> monograph guidelines initially and after an extended storage period.

### SUMMARY OF THE INVENTION

The present invention seeks to provide an improved oral compressed solid composition that disintegrates rapidly and provides a rapid dissolution of magnesium salt after oral administration. A rapidly disintegrating and rapidly dissolving composition comprising one or more magnesium salts, one or more hydrophilic polymers, one or more disintegrants, optionally one or more surfactants, optionally one or more glidants, optionally one or more fillers, and optionally one or more lubricants is provided. One or more other common pharmaceutical excipients can be included. The compressed solid composition may optionally be enclosed within a capsule shell as an over-encapsulated tablet or pill where the capsule shell serves only as an aesthetic trade dress. The tablet may optionally be coated with a rapidly dissolving coating. The oral formulation provides a substantially stable dissolution profile under USP <711> conditions for the magnesium salt over an extended period of storage. The compressed composition excludes a therapeutic amount of any therapeutically active agent other than a magnesium salt.

The magnesium salt in the tablet dissolves rapidly, according to the USP <711> guidelines for dissolution of magnesium oxide tablets, in gastric juice. The composition exhibits a stable shelf life, such that its dissolution properties do not change significantly over time, for example at least one year or at least two years as estimated by storage at 40° C. and 75% relative humidity. As such, the composition will continue to meet the USP <711> guidelines for dissolution even after an extended storage period in a seal container-enclosure system.

In one embodiment, exclusion of an added cellulose-based excipient, such as microcrystalline cellulose, from the formulation is at least partially, or primarily, responsible for the extended shelf life. Even so, the tablet composition of the invention can include small amounts (<5%) of the cellulose-based excipient.

In another embodiment, exclusion of excess or added moisture during processing and storage is at least partially, or primarily, responsible for the extended shelf life. A first process for preparing the tablets employs anhydrous conditions, thereby limiting exposure of the tablet components to moisture. Even so, residual moisture content in the tablets up to about 4%, as determined by loss on drying at 105° C., may be tolerated during the manufacture and storage of the solid composition depending upon the formulation used.

A range of suitable hydrophilic polymers can be used. In one embodiment, a combination of two or more different hydrophilic polymers, such as a polyalkylene glycol having a molecular weight in the range of 3000-8000 and a polyoxyethylene-polyoxypropylene block copolymer, is used as the hydrophilic polymer.

One aspect of the invention provides a rapidly dissolving solid oral composition comprising:
one or more magnesium salts;
one or more hydrophilic polymers;
one or more disintegrants;
optionally one or more surfactants;
optionally one or more glidants;
optionally one or more fillers; and
optionally one or more lubricants;
wherein the composition provides a substantially stable dissolution profile according to USP <711> and the magnesium oxide tablet monograph for the one or more magnesium salts for a period of at least 2 months when the composition is stored under pharmaceutically acceptable storage conditions such as 40° C. and 75% relative humidity in a sealed container-enclosure system.

Specific embodiments of the invention include those wherein: 1) the magnesium salt is MgO or Mg(OH)₂; 2) the one or more hydrophilic polymers is a combination of polymers; 3) the disintegrant is selected from the group consisting of: crospovidone, croscarmellose sodium, sodium starch glycolate, and low-substituted (which according to the USP/NF specification for L-HPC is HPC having 5-16% by wt. of hydroxypropyl groups) hydroxypropyl cellulose; 4) the one or more hydrophilic polymers is selected from the group consisting of: polyethylene glycol, poloxamer, and povidone; 5) the storage period is at least two months at 40° C. and 75% relative humidity; 6) the composition is a compressed composition; 7) the composition is included as a tablet in a capsule dosage form; 8) the composition is prepared by dry granulation; 9) the composition is prepared by direct compression; 10) the magnesium salt is a sparingly soluble, slightly soluble, very slightly soluble, practically insoluble or insoluble salt; 11) the magnesium salt is the only component present in a therapeutically active amount; 12) the composition comprises less than 7.5% water; 13) the composition comprises less than 5.5% water; 14) the composition comprises less than 4% water; and/or 15) the composition excludes added microcrystalline cellulose.

The invention also provides a solid oral dosage form comprising a compressed composition as defined herein.

The invention also provides a method of preparing a compressed solid composition comprising magnesium salt, one or more hydrophilic polymers, one or more disintegrants, optionally one or more surfactants, optionally one or more glidants, optionally one or more fillers, and optionally one or more lubricants, wherein the composition provides a substantially stable dissolution profile according to USP <711> for the one or more magnesium salts for a period of at least 2 months when the composition is stored under pharmaceutically acceptable storage conditions such as 40° C. and 75% relative humidity, the method comprising the steps of: 1) admixing the magnesium salt with one or more hydrophilic polymers, one or more disintegrants, optionally with surfactants, fillers, and/or lubricants in a suitable powder mixer; 2) powders are blended to achieve a homogenous mixture; 3) blended mixture is then compressed into magnesium tablets. The magnesium tablets may then be packaged. This process is known as direct compression. Magnesium tablets may also be prepared by dry granulation techniques known to those skilled in the arts, i.e. slugging or roller compaction. Slugging operations include the blending of the magnesium salt with the other components of the composition and then slugging or tabletting this mixture into compacts that are subsequently ground or milled into granules. The granules are then re-compressed into the final tablet product. Roller compaction produces agglomerates of a powder blend containing a magnesium salt by compressing the powders between two counter-rotating rolls. The compacted material is then ground or milled into granules that are subsequently compressed into tablets. Direct compression and dry granulation are anhydrous process methods since they do not require the addition of or use of water in the production of the compressed dosage form containing the magnesium salts and associated excipients. Wet granulation using non-aqueous solvents such as alcohols, ethyl acetate, chloroform, methylene chloride, acetone, or the like, could be employed to produce granulations of powders for subsequent compaction into tablets.

These and other aspects of this invention will be apparent upon reference to the following detailed description, examples, claims and attached figures.

### BRIEF DESCRIPTION OF THE FIGURES

The following drawings are given by way of illustration only, and thus are not intended to limit the scope of the present invention. The USP27/NF22 monograph for magnesium oxide tablets specifies that the dissolution method is to be executed using the general conditions of chapter <711> with 900 mL of 0.1 N hydrochloric acid, a paddle rotation speed of 75 RPM, and a sampling timepoint of 45 minutes.

FIG. 1 depicts the dissolution profiles for magnesium when released from a lot of MAGnesium-Oxide™ tablets of GENESIS PRODUCTS, INC. under the conditions of the magnesium oxide tablet monograph and USP <711>. The dissolution of Mg from tablets stored in unopened containers at 40° C. and 75% relative humidity for 2 months is provided.

FIG. 2 depicts the dissolution profiles for magnesium when released from a lot of magnesium oxide tablets of CYPRESS PHARMACEUTICAL INC. under the conditions of the magnesium oxide tablet monograph and USP <711>. The dissolution of Mg from tablets stored in unopened containers at 40° C. and 75% relative humidity for 2 months is provided.

FIG. 3 depicts the dissolution profiles for magnesium when released from a lot of YOURLIFE™ Natural magnesium oxide tablets of LEINER HEALTH PRODUCTS under the conditions of the magnesium oxide tablet monograph and USP <711>. The dissolution of Mg from tablets stored in unopened containers at 40° C. and 75% relative humidity for 2 months is provided.

FIG. 4 depicts the influence of packaging and storage on the dissolution of magnesium from pure magnesium oxide. Samples were stored at 40° C. and 75% relative humidity for up to 2 months. The samples were packaged in loosely capped (Open) high density polyethylene bottles, bottles with tight caps and aluminum induction seals (Sealed), or bottles with tight caps, an aluminum induction seal, and containing a silica gel desiccant. Dissolution testing was performed using the method specifications of USP <711> for magnesium oxide tablets.

FIG. 5 depicts the influence of various excipients on the dissolution of magnesium from binary mixtures of MgO and excipient are evaluated under magnesium oxide tablet USP <711> conditions.

FIG. 6 depicts a chart of the influence of storage upon the MgO dissolution profile of the formulation of Example 4. The dissolution of Mg from tablets stored in unopened containers at 40° C. and 75% relative humidity for 2 months is provided.

FIG. 7 depicts a chart of the influence of storage upon the MgO dissolution profile of the formulation of Example 5. The dissolution of Mg from tablets stored in unopened containers at 40° C. and 75% relative humidity for 2 months is provided.

FIG. 8 depicts a chart of the influence of storage upon the MgO dissolution profile of the formulation of Example 6. The dissolution of Mg from tablets stored in unopened containers at 40° C. and 75% relative humidity for 2 months is provided.

FIG. 9 depicts a chart of the influence of storage upon the MgO dissolution profile of the formulation of Example 7. The dissolution of Mg from tablets stored in unopened containers at 40° C. and 75% relative humidity for 2 months is provided.

FIG. 10 depicts a chart of the influence of storage upon the MgO dissolution profile of the formulation of Example 8. The dissolution of Mg from tablets stored in unopened containers at 40° C. and 75% relative humidity for 2 months is provided.

FIG. 11 depicts a chart of the influence of storage upon the MgO dissolution profile of the formulation of Example 9. The dissolution of Mg from tablets stored in unopened containers at 40° C. and 75% relative humidity for 2 months is provided.

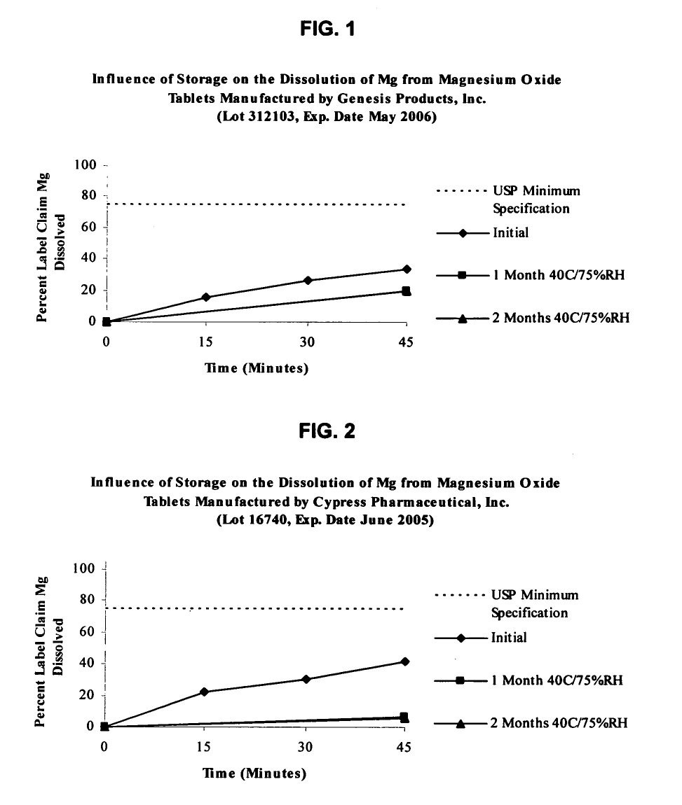

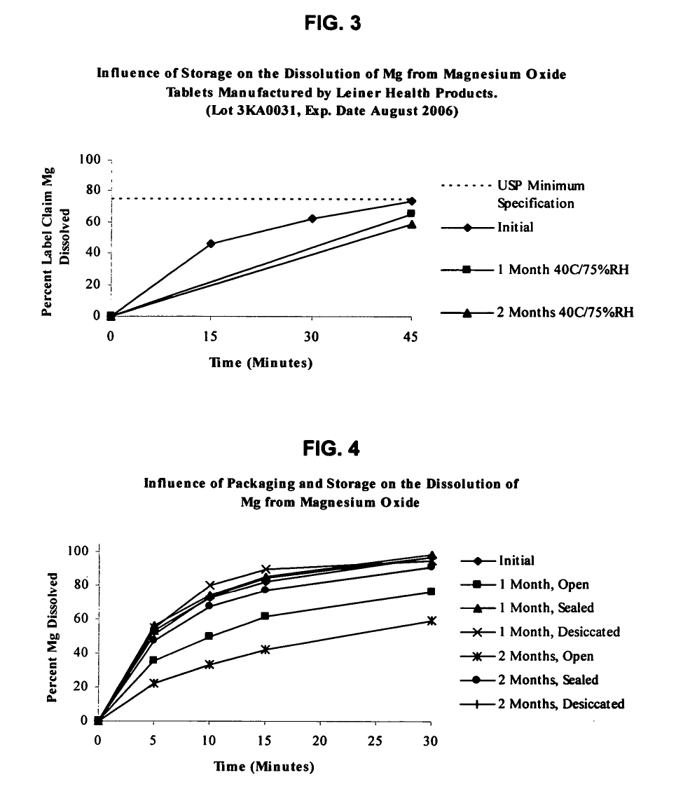

### DETAILED DESCRIPTION OF THE INVENTION

A magnesium salt-containing formulation of the invention provides unexpected advantages over related magnesium salt-containing formulations. The compressed tablet formulation provides a substantially stable dissolution profile even after a period of storage of 2 months under pharmaceutically acceptable storage conditions (40° C. and 75% relative humidity).

The magnesium salt can be an organic or inorganic salt of magnesium. Exemplary salts include magnesium oxide, magnesium hydroxide, magnesium chloride, magnesium gluconate, magnesium aspartate, magnesium citrate, magnesium glycinate, magnesium carbonate, magnesium amino acid chelate, magnesium ascorbate, magnesium α-keto-glutarate, magnesium taurinate, magnesium sulfate, magnesium tartrate, magnesium fumarate, magnesium maleate, magnesium lactate, magnesium stearate, magnesium phosphate (dibasic), magnesium oxalate dihydrateand others known to those of ordinary skill in the art. The present invention also includes all of the non-hydrated, hydrated, and polymorphic forms of the above-identified salts. Suppliers often use different processes for making magnesium salts. Accordingly, MgO from one supplier will likely have a different particle size, bulk density and/or porosity than MgO from another suppler. The present invention includes magnesium salts available in any pharmaceutically acceptable particle size range. The magnesium salts of the invention will have a bulk density and/or porosity that is suitable for use in the formulation and process of the invention.

The different magnesium salts are known to have different water solubility.

| | Solubility in Neutral Water | USP 27/NF22 |
|---|---|---|
| Magnesium Salt | (g/100 mL) | Solubility |
| MgO | 0.00062 | Practically Insoluble |
| Mg(OH)₂ | 0.0012 | Practically Insoluble |
| MgF₂ | 0.0002 | Practically Insoluble |
| MgCl₂ | 166.3 | Very Soluble |
| MgSO₄ | 123.1 | Very Soluble |
| Mg Citrate L-Carnitine | >50 | Very Soluble |
| Mg Carbonate | <0.01 | Practically Insoluble |
| Mg Gluconate | 16 | Freely Soluble |
| Mg Lactate | 5.2 | Soluble |

The efficiency of absorption (fractional absorption) of a magnesium salt may depend upon its solubility in intestinal fluids, as well as on the amount digested. Salts with high solubility, e.g., magnesium citrate, may be more efficiently absorbed than salts with poor solubility, e.g., magnesium oxide. The counter anion of the magnesium salt may additionally influence its absorption.

Magnesium oxide (sometimes called magnesia) is formed commercially by heating magnesite (MgCO₃) to 600-800° C., which drives off most of the CO₂. The thermal decomposition of magnesium chloride, magnesium sulfate, magnesium sulfite, and nesquehonite will also yield MgO. It also occurs naturally as the mineral periclase. Calcining magnesium hydroxide or the mineral magnesite that is obtained by liming from seawater can produce the mineral periclase. The collected MgO is then purified. The grade of magnesium salt, esp. magnesium oxide, need not be limited to any particular grade. Magnesium oxide (MgO) is available in different grades that are classified according to density or mode of heat curing. The bulk density of magnesium oxide typically ranges from 0.13 g/mL to 0.5 g/mL with some granulated grades having a bulk density near 1 g/mL. The surface area of certain types of MgO has been reported to range from 28-35 m²/g. Heavy (high density) MgO is a lower porosity material prepared by higher heat treatment; whereas light MgO is a higher porosity material prepared by lower heat treatment. According to 21 U.S.C. 184.1431, heating magnesium salts under moderate conditions (400 to 900° C. for a few hours) produces light magnesium oxide, and heating the salts under more rigorous conditions (1200° C. for 12 hours) produces heavy magnesium. U.S. Pharmacopoeia specifications for both light and heavy grades of MgO are listed under the Magnesium Oxide monograph. MgO can be prepared with different bulk densities by spray drying or granulation. Both grades of MgO are suitable for use in the tablet of the invention; however, the higher density grades of MgO are particularly suitable for use. The higher density grades of MgO are designed to facilitate direct compression and/or dry granulation processing by possessing better mass flow properties and densities more similar to those of the other materials in the tablet mass to facilitate better mixing.

The following tables include characterization data for MgO obtained from different suppliers.

Suppliers of Pharmaceutical Grade Magnesium Oxide

| Supplier Number | Company | Address |
|---|---|---|
| 1 | Mallinckrodt, Inc. | St. Louis, MO 63134 |
| 2 | Particle Dynamics | St. Louis, MO 63144 |
| 3 | Tomita Pharmaceutical Co., | Naruto-City, Tokushima |
| | Ltd. | 771-0360 Japan |

Characterization of Magnesium Oxide from Supplier 1

| Lot | D07145 | D03337 |
|---|---|---|
| Grade | Light, USP | Heavy, USP |
| Bulk Density | 0.15 g/mL | 0.34 g/mL |
| Tapped Density | 0.19 g/mL | 0.48 g/mL |

Characterization of Magnesium Oxide from Supplier 2

| Lot | 500 |
|---|---|
| Grade | Heavy, USP |
| Bulk Density | 0.52 g/mL |
| Tapped Density | 0.69 g/mL |

Characterization of Magnesium Oxide from Supplier 3

| Lot | E10807 | E91206 |
|---|---|---|
| Grade | (S), Granular, USP | (SF), Granular, USP |
| Bulk Density | 1.05 g/mL | 1.08 g/mL |
| Tapped Density | 1.10 g/mL | 1.15 g/mL |

The magnesium salt used in the present tablet has a defined solubility in water. The magnesium salt can be “very soluble”, “freely soluble”, “soluble”, “sparingly soluble”, “slightly soluble”, “very slightly soluble”, and “practically insoluble” or “insoluble” as such terms are defined in the USP 27/NF 22 as follows:

| | Parts of Solvent Required for 1 Part of |
|---|---|
| Term | Solute |
| Very soluble | <1 |
| Freely soluble | 1-10 |
| Soluble | 10-30 |
| Sparingly soluble | 30-100 |
| Slightly soluble | 100-1,000 |
| Very slightly soluble | 1,000-10,000 |
| Practically insoluble or insoluble | Over 10,000 |

The present invention is particularly suited for “sparingly soluble”, “slightly soluble”, “very slightly soluble”, and “practically insoluble” or “insoluble” magnesium salts.

The oral formulation of the invention can be changed according to the guidelines herein to permit reproducible delivery of any pharmaceutically acceptable magnesium salt. For example, the tablet will provide a substantially stable dissolution profile for the magnesium salt even after extended storage of the tablet under pharmaceutically acceptable conditions.

FIG. 4 depicts dissolution profiles for each MgO as determined under USP <711> when stored at 40° C. and 75% relative humidity. The conditions for storage were as follows: 1) loosely capped (Open) high density polyethylene bottles (-▪-: 1 month storage; -*-; 2 months); 2) bottles with tight caps (Sealed) and aluminum induction seals (-▴-: 1 month; -●-: 2 months); or 3) bottles with tight caps, an aluminum induction seal, and containing a silica gel desiccant (Desiccated; -x-: 1 month; -+-: 2 months). The three methods used to package the formulation are detailed in Example 10. The data demonstrate suitable dissolution for MgO when protected from moisture.

According to the method of the invention, a preferred packaging material and condition minimizes exposure of the formulation to excessive or added moisture. The packaging material is typically a container that holds the present formulation and is in direct contact with it. The General Notices to USP 27/NF 22 describes the immediate container as that which is in direct contact with the article at all times with the closure, or cap, being part of the container. Furthermore, well-closed containers protect the contents from extraneous solids and from loss of the article under the ordinary or customary conditions of handling, shipment, storage, and distribution. Tight containers protect the contents from contamination by extraneous liquids, solids, or vapors, from loss of the article, and from efflorescence, deliquescence, or evaporation under the ordinary or customary conditions of handling, shipment, storage, distribution, and is capable of tight re-closure. Lastly, a hermetic container is substantially impervious to air or any other gas under the ordinary or customary conditions of handling, shipment, storage, and distribution.

21 C.F.R. 211.94 provides information that is to be used when evaluating containers and closures to be used with drug products. Any container and closure found to be suitable under such guidelines are suitable for storing the compressed composition of the invention. The containers and closures should not be reactive, additive, or absorptive so as to alter the safety, identity, strength, quality, or purity of the magnesium salt beyond the official or established requirements. The container closure systems should provide adequate protection against foreseeable external factors in storage and use that can cause deterioration or contamination of the magnesium salt. The containers and closures should be clean and, optionally, sterilized and processed to remove pyrogenic properties to ensure that they are suitable for their intended use.

For example, the sealed bottles are preferred since they are tight containers. Criteria for establishing that a bottle is sealed under various different conditions are set forth in U.S.P. <671> Containers-Permeation. In that method, the moisture permeability of container-closure system is determined. U.S.P. <661> details several methods for the characterization of materials used in containers and/or closures. The formulation of the invention is preferably packaged in a sealed container that minimizes entry of moisture from the exterior of the container to the interior of the container. The seal for the enclosure, e.g. lid or cap, is generally airtight. Accordingly, a tight seal is used for the enclosure of the container.

The container of the invention can be any container typically used in the pharmaceutical industry to store a solid formulation and maintain it in a sealed environment. A container can be made of plastic, glass, and/or metal. A suitable container can be a bottle, vial, jar, ampule, single dose container, multi-dose container, or other packaging system known to those of ordinary skill in the art.

A sealed container-closure system used according to the invention might permit passage of air and/or moisture; however, a suitably sealed container-closure system will minimize such passage. For example, the formulation of the invention was placed in a container-enclosure system according to Example 10. Even though some moisture was able to permeate through the sealed container-closure system, the present formulation displayed a substantially stable dissolution profile, but the commercial prior art formulations tested did not.

Although glass and metal containers and closures may be used to package the invention, the use of polymeric containers and closures is much more prevalent in the food, nutritional, and/or pharmaceutical arenas. Suitable polymeric packaging materials for use as the container and enclosure are described in 21 C.F.R. 177. Such materials are described as safe for food contact. In addition, the materials will be stable to pharmaceutically acceptable storage conditions. It should be noted that materials used to evaluate the storage stability of the present formulation under accelerated stability testing conditions need not be but can be the same as materials in which the formulation is actually stored for marketing.

It should be noted, that samples of the present formulation were packaged in sealed containers similar to those found in the prior art commercial formulations. Samples of unopened commercial formulations were evaluated side-by-side to the present formulations. The present formulations were found to retain a stable dissolution profile but prior art commercial formulations were not when stored under the same accelerated stability testing conditions in similar types of container-enclosure systems. The following table describes the immediate containers of several prior art commercial formulations.

| Product | Manufacturer | Packaging Description |
|---|---|---|
| (Magnesium Source) | | |
| Mag-Tab ® SR | Schering Corporation | White High Density Polyethylene (HDPE) |
| (Magnesium L- | | bottle, 35 mm closure, paper laminate taper |
| Lactate Dihydrate | | evident seal |
| Maginex ™ | Verla-Pharm | Blister packed, plastic on foil |
| (Magnesium L- | Arzneimittel | |
| Aspartate HCl) | | |
| MAGnesium- | Genesis Products, Inc. | White HDPE bottle, 35 mm child resistant |
| Oxide ™ | | closure (CRC), paper and foil laminate taper |
| (Magnesium Oxide) | | evident seal |
| Magnesium Oxide | Cypress | White HPDE bottle, 35 mm CRC closure, |
| 400 mg | Pharmaceuticals, Inc. | paper and foil laminate taper evident seal |
| (Magnesium Oxide) | | |
| YourLife ™ Natural | Leiner Health | White HDPE bottle, 35 mm non-CRC closure, |
| Magnesium | Products, Inc. | paper laminate taper evident seal |
| (Magnesium Oxide) | | |
| High Potency | Nature's Bounty | Dark Amber, Polyethylene Terphthalate (PET) |
| Magnesium | | bottle, 38 mm closure, paper and foil laminate |
| (Magnesium Oxide) | | tamper evident seal |
| Beech Beelith | Beech Pharmaceuticals | Amber HDPE bottle, 35 mm CRC closure, |
| Magnesium | | paper and foil laminate tamper evident seal |
| Supplement | | |
| (Magnesium Oxide) | | |
| MAOX | Kenneth A. Manne | White HDPE bottle, 38 mm non-CRC closure, |
| (Magnesium Oxide) | Company | paper and foil laminate taper evident seal |
| Nature's Made | Nature's Made | Amber PET bottle, 35 mm non-CRC closure, |
| (Magnesium Oxide) | Nutritional Products | paper and foil laminate tamper evident seal |

Polymer resins used as materials in pharmaceutical packaging have different moisture vapor transmission rates (MVTR). The following table lists the MVTR of several polymers used in the packaging of food, nutritional, and pharmaceutical products. Information obtained from EVAL Company of America Technical Bulletin No. 110 REV.07-00.

| | MVTR | MVTR |
|---|---|---|
| | (40° C., 90% R.H.) | (40° C., 90% R.H.) |
| Structural Material | g · 25 μ/m²/24 Hrs. | g · mil/100 in²/24 Hrs. |
| High Density | 5.9 | 0.38 |
| Polyethylene (HDPE) | | |
| Polypropylene (PP) | 10.7 | 0.69 |
| Low Density | 17.7 | 1.14 |
| Polyethylene (LDPE) | | |
| Polyethylene | 20.2 | 1.3 |
| Terephthalate (PET) | | |
| Rigid Polyvinyl Chloride | 46.5 | 3.0 |
| (PVC) | | |
| Polystyrene (PS) | 131.8 | 8.5 |
| Polycarbonate | 170.5 | 11.0 |

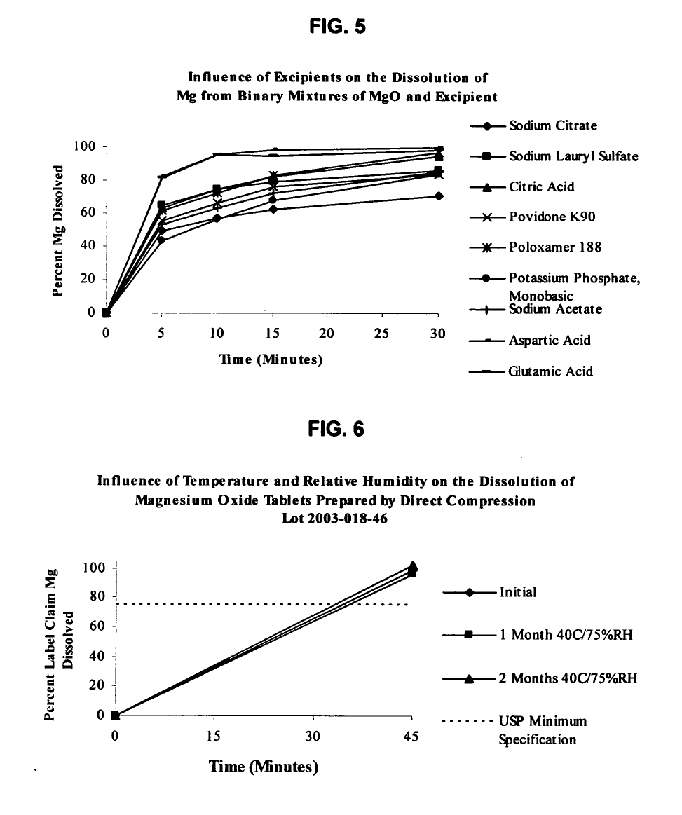

The impact that different excipients have upon dissolution of MgO was evaluated. A binary mixture of heavy grade magnesium oxide, USP was prepared with each of the listed excipients. The dissolution of magnesium from a sample of the blend was then evaluated using the dissolution conditions outlined in the magnesium oxide tablet monograph USP <711>. The results from FIG. 5 indicate that the inclusion of excipients can impact the dissolution of magnesium from the oxide salt of magnesium. Some excipients (organic acids, poloxamer) will facilitate dissolution while other excipients (potassium phosphate, sodium citrate) will decrease dissolution.

Thus, the invention provides a compressed solid composition that provides a rapid dissolution of MgO according to the specifications of the magnesium oxide tablet monograph and the general USP <711> dissolution monograph. However, unlike the prior art formulations, the composition of the invention provides a substantially stable dissolution profile for the magnesium salt, esp. MgO. By “substantially stable dissolution profile” is meant the dissolution characteristics of the formulation will not change significantly upon extended storage under pharmaceutically acceptable storage conditions. By “will not change significantly” is meant the dissolution profile for the magnesium salt will still meet the individual monograph and USP <711> criteria for rapid dissolution for the respective salt and respective compressed tablet if an individual monograph exists. By “extended storage” is meant a period of time exceeding 2 months under pharmaceutically acceptable storage conditions.

By “pharmaceutically acceptable storage conditions” is meant storage at 25° C. and 60% relative humidity (RH) in a tight, sealed container-enclosure system. The storage conditions employed herein (40° C. and 75% RH) in evaluating formulations are used in the pharmaceutical industry to conduct accelerated stability testing of formulations such that a period of 2 months under these conditions is generally accepted to equal a period of 1 year under pharmaceutically acceptable storage conditions (25° C. and 60% relative humidity).

Dry granulation (BLA-7ST 2003-010-35) and direct compression (BLA-36DC 2003-010-34) were compared side-by-side using two substantially equivalent formulations, the only key difference being the method of preparation of the tablet. Both methods are suitable for preparing a tablet according to the invention. It was unexpectedly discovered that dry granulation provides a tablet having an even more rapid dissolution of MgO. In one embodiment, cellulose-based excipients were eliminated from the formulation and tablets were prepared by direct compression according to Examples 1 and 2. It was unexpectedly discovered that elimination (preclusion) of an added cellulose-based excipient from the formulation improved tablet performance in terms of stability of the MgO dissolution profile.

Although not necessary, the formulation of the present invention may include a adsorbent, acidifying agent, antiadherent, binder, antioxidant, buffering agent, diluent (filler), direct compression excipient, alkalizing agent, bulking agent, colorant, plasticizer, stabilizer, flavor, sweetener, disintegrant, glidant, lubricant, opaquant, polishing agent, fragrance, surfactant and/or other excipients known by those of ordinary skill in the art for use in formulations, or a combination thereof.

As used herein, the term “adsorbent” is intended to mean an agent capable of holding other molecules onto its surface by physical or chemical (chemisorption) means. Such compounds include, by way of example and without limitation, powdered and activated charcoal and other materials known to one of ordinary skill in the art.

As used herein, the term “acidifying agent” is intended to mean a compound used to provide an acidic medium for product stability. Such compounds include, by way of example and without limitation, acetic acid, acidic amino acids, citric acid, fumaric acid and other alpha hydroxy acids, hydrochloric acid, ascorbic acid, phosphoric acid, sulfiric acid, tartaric acid and nitric acid and others known to those of ordinary skill in the art.

As used herein, the term “antiadherent” is intended to mean an agent that prevents the sticking of solid dosage formulation ingredients to punches and dies in a tableting machine during production. Such compounds include, by way of example and without limitation, magnesium stearate, talc, calcium stearate, glyceryl behenate, PEG, hydrogenated vegetable oil, mineral oil, stearic acid and other materials known to one of ordinary skill in the art.

As used herein, the term “binder” is intended to mean a substance used to cause adhesion of powder particles in solid dosage formulations. Such compounds include, by way of example and without limitation, acacia, alginic acid, carboxymethylcellulose sodium, compressible sugar (e.g., NuTab), ethylcellulose, gelatin, liquid glucose, methylcellulose, povidone and pregelatinized starch. Other exemplary binders include acacia, tragacanth, gelatin, starch, cellulose materials such as methyl cellulose and sodium carboxy methyl cellulose, alginic acids and salts thereof, polyethylene glycol, guar gum, polysaccharide, bentonites, sugars, invert sugars, poloxamers (Lutrol™ F68, Lutrol™ F127), collagen, albumin, gelatin, cellulosics in nonaqueous solvents, combinations thereof and others known to those of ordinary skill in the art. Other binders include, for example, polypropylene glycol, polyoxyethylene-polypropylene copolymer, polyethylene esters, polyethylene oxide, combinations thereof and other materials known to one of ordinary skill in the art.

As used herein, the term “diluent” or “filler” is intended to mean an otherwise inert substance used as a filler to create the desired bulk, flow properties, and compression characteristics in the preparation of solid dosage forms. Such compounds include, by way of example and without limitation, dibasic calcium phosphate, kaolin, lactose, dextrose, magnesium carbonate, sucrose, mannitol, microcrystalline cellulose, powdered cellulose, precipitated calcium carbonate, sorbitol, and starch and other materials known to one of ordinary skill in the art.

As used herein, the term “direct compression excipient” is intended to mean a compound used in compressed solid dosage forms. Such compounds include, by way of example and without limitation, dibasic calcium phosphate (e.g., Ditab) and other materials known to one of ordinary skill in the art.

As used herein, the term “antioxidant” is intended to mean an agent that inhibits oxidation and thus is used to prevent the deterioration of preparations by the oxidative process. Such compounds include, by way of example and without limitation, acetone, potassium metabisulfite, potassium sulfite, ascorbic acid, ascorbyl palmitate, citric acid, butylated hydroxyanisole, butylated hydroxytoluene, hypophophorous acid, monothioglycerol, propyl gallate, sodium ascorbate, sodium citrate, sodium sulfide, sodium sulfite, sodium bisulfite, sodium formaldehyde sulfoxylate, thioglycolic acid, EDTA, pentetate, and sodium metabisulfite and others known to those of ordinary skill in the art.

As used herein, the term “buffering agent” is intended to mean a compound used to resist change in pH upon dilution or addition of acid or alkali. Such compounds include, by way of example and without limitation, acetic acid, sodium acetate, adipic acid, benzoic acid, sodium benzoate, boric acid, sodium borate, citric acid, glycine, maleic acid, monobasic sodium phosphate, dibasic sodium phosphate, HEPES, lactic acid, tartaric acid, potassium metaphosphate, potassium phosphate, monobasic sodium acetate, sodium bicarbonate, tris, sodium tartrate and sodium citrate anhydrous and dihydrate and others known to those of ordinary skill in the art.

As used herein, a fragrance is a relatively volatile substance or combination of substances that produces a detectable aroma, odor or scent. Exemplary fragrances include those generally accepted as FD&C.

As used herein, the term “glidant” is intended to mean an agent used in solid dosage formulations to promote flowability of the solid mass. Such compounds include, by way of example and without limitation, colloidal silica, cornstarch, talc, calcium silicate, magnesium silicate, colloidal silicon, tribasic calcium phosphate, silicon hydrogel and other materials known to one of ordinary skill in the art.

As used herein, the term “lubricant” is intended to mean a substance used in solid dosage formulations to reduce friction during compression. Such compounds include, by way of example and without limitation, calcium stearate, magnesium stearate, PEG, talc, mineral oil, stearic acid, and zinc stearate and other materials known to one of ordinary skill in the art.

As used herein, the term “opaquant” is intended to mean a compound used to render a coating or composition opaque. Opaquants may be used alone or in combination with a colorant. Such compounds include, by way of example and without limitation, titanium dioxide, talc and other materials known to one of ordinary skill in the art.

As used herein, the term “polishing agent” is intended to mean a compound used to impart an attractive sheen to solid dosage forms. Such compounds include, by way of example and without limitation, carnauba wax, white wax and other materials known to one of ordinary skill in the art.

As used herein, the term “disintegrant” is intended to mean a compound used in solid dosage forms to promote the disruption of the solid mass into smaller particles which are more readily dispersed or dissolved. Exemplary disintegrants include, by way of example and without limitation, starches such as corn starch, potato starch, pre-gelatinized and modified starches thereof, sweeteners, clays, bentonite, microcrystalline cellulose (e.g., Avicel), carboxymethylcellulose calcium, croscarmellose sodium, alginic acid, sodium alginate, cellulose polyacrilin potassium (e.g., Amberlite), alginates, sodium starch glycolate, gums, agar, guar, locust bean, karaya, pectin, tragacanth, crospovidone and other materials known to one of ordinary skill in the art.

As used herein, the term “stabilizer” is intended to mean a compound used to stabilize the therapeutic agent against physical, chemical, or biochemical process which would reduce the therapeutic activity of the agent. Suitable stabilizers include, by way of example and without limitation, albumin, sialic acid, creatinine, glycine and other amino acids, niacinamide, sodium acetyltryptophonate, zinc oxide, sucrose, glucose, lactose, sorbitol, mannitol, glycerol, polyethylene glycols, sodium caprylate and sodium saccharin and other known to those of ordinary skill in the art.

The formulation of the invention can also include oils, for example, fixed oils, such as peanut oil, sesame oil, cottonseed oil, corn oil and olive oil; fatty acids, such as oleic acid, stearic acid and isostearic acid; and fatty acid esters, such as ethyl oleate, isopropyl myristate, fatty acid glycerides and acetylated fatty acid glycerides. It can also include alcohols, such as ethanol, isopropanol, hexadecyl alcohol, glycerol and propylene glycol; glycerol ketals, such as 2,2-dimethyl-1,3-dioxolane-4-methanol; ethers, such as poly(ethylene glycol) 450; petroleum hydrocarbons, such as mineral oil and petrolatum; or mixtures thereof.

Soaps and synthetic detergents may be employed as surfactants. Suitable detergents include cationic detergents and surfactants, for example, polyamines and their salts, quaternary ammonium salts, and amine oxides, alkyl dimethyl substituted halides, dimethyl dialkyl ammonium halides, dimethyl substituted benzene-methanaminium halides, dodecyltrimethylammonium halides, trimethyltetradecylammonium halides, hexadecyltrimethylammonium halides, alkyl pyridinium halides, and alkylamine acetates; anionic detergents and surfactants for example, sulfonic acid salts, alcohol sulfates, alkylbenzene sulfonates, phosphoric acid esters, and carboxylic acid salts, sodium lauryl sulfate, alkyl, aryl, and olefin sulfonates, alkyl olefin, ether and monoglyceride sulfates, and sulfosuccinates; nonionic surfactants and detergents, for example, polyoxyethylenated alkylphenols, alcohol ethoxylates, alkylphenol ethoxylates, and alkanolamides, fatty amine oxides, fatty acid alkanolamides, and poly(oxyethylene)-block-poly(oxypropylene) copolymers, glycerol monooleate, polysorbate 20, polysorbate 21, polysorbate 40, polysorbate 60, polysorbate 61, polysorbate 65, polysorbate 80, polysorbate 81, polysorbate 85, polysorbate 120, polyvinyl alcohols, and sorbitan esters; amphoteric detergents, for example, alkyl β-aminopropionates, and 2-alkylimidazoline quaternary ammonium salts; synthetic or naturally occurring phosphatide; others known to those of ordinary skill in the art; and combinations thereof.

Hydrophilic polymers can be used to improve the performance of the solid oral composition. Exemplary hydrophilic polymers suitable for use in a magnesium salt containing composition included in, for example, Remington's Pharmaceutical Sciences, 18th Edition, Alfonso R. Gennaro (editor), Mack Publishing Company, Easton, Pa., 1990, pp. 291-294; Alfred Martin, James Swarbrick and Arthur Commarata, Physical Pharmacy. Physical Chemical Principles in Pharmaceutical Sciences, 3rd edition (Lea & Febinger, Philadelphia, Pa., 1983, pp. 592-638); A. T. Florence and D. Altwood, (Physicochemical Principles of Pharmacy, 2nd Edition, MacMillan Press, London, 1988, pp. 281-334; R. C. Rowe, P. J. Sheskey, and P. J. Weller (eds.), Handbook of Pharmaceutical Excipients, 4^{th} edition (Pharmaceutical Press, London, 2003. The entire disclosures of the references cited herein are hereby incorporated by reference. Still other suitable polymers include water-soluble natural polymers, water-soluble semi-synthetic polymers (such as the water-soluble derivatives of cellulose) and water-soluble synthetic polymers. The natural polymers include polysaccharides such as inulin, pectin, algin derivatives (e.g. sodium alginate) and agar, and polypeptides such as casein and gelatin. The semi-synthetic polymers include cellulose derivatives such as methylcellulose, hydroxyethylcellulose, hydroxypropyl cellulose, their mixed ethers such as hydroxypropyl methylcellulose and other mixed ethers such as hydroxyethyl ethylcellulose and hydroxypropyl ethylcellulose, hydroxypropyl methylcellulose phthalate and carboxymethylcellulose and its salts, especially sodium carboxymethylcellulose. The synthetic polymers include polyoxyethylene derivatives (polyethylene glycols) and polyvinyl derivatives (polyvinyl alcohol, polyvinylpyrrolidone and polystyrene sulfonate) and various copolymers of acrylic acid (e.g. carbomer). Other natural, semi-synthetic and synthetic polymers not named here which meet the criteria of water solubility, pharmaceutical acceptability and pharmacological inactivity are likewise considered to be within the ambit of the present invention.

It should be understood, that compounds used in the art of pharmaceutical formulations generally serve a variety of functions or purposes. Thus, if a compound named herein is mentioned only once or is used to define more than one term herein, its purpose or function should not be construed as being limited solely to that named purpose(s) or function(s).

Since some of the formulations herein are subject to modification by moisture, a solid composition formulation according to the invention can be prepared under anhydrous conditions. In one embodiment, the ingredients of the composition are mixed under anhydrous conditions. Anhydrous process conditions are typically defined as those processes that do not require the addition of water or moisture beyond that found in the ambient atmosphere or inherent the unprocessed raw materials. Direct compression and dry granulation are examples of anhydrous process conditions used to prepare dosage forms. Other anhydrous processes include melt processing or solvent processing where the solvent does not contain a substantial quantity of water. Substantially anhydrous conditions (less than 60% RH) can be used for storage conditions to further improve the stability of the formulation. In a specific embodiment of the invention, a formulation is prepared by a process not requiring water, i.e., a process where water is not purposefully added to the formulation or a substantially anhydrous process, and the resulting formulation was stored in a sealed container-enclosure system such that the interior of the container was substantially anhydrous even though the exterior was not (it was exposed to 75% RH).

When the compressed solid composition is enclosed within a capsule shell, one or more units of the composition can be included. The shell is intended for rapid dissolution or disintegration in acidic medium. Suitable shell compositions include hard or soft shell capsules made of any material or combination of materials adapted for dissolution and/or disintegration in the upper gastrointestinal tract after oral administration to a subject. The capsule shell can comprise hard gelatin, soft gelatin, starch, or other suitable materials for molded for the intended use and oral ingestion.

When the compressed solid composition is enclosed within a water (which can be acidic, neutral or alkaline) soluble and/or erodible coating, the coating will generally comprise an inert and non-toxic material that is at least partially, and generally substantially completely, soluble or erodible in an aqueous environment of use. The coating will be adapted for dissolution and/or erosion in the buccal cavity and/or upper GI tract, such as the stomach, duodenum, jejunum or upper small intestines. The inert water soluble and/or erodible coat covering the composition is made of synthetic or natural material(s). These coatings may be applied from either aqueous or non-aqueous solvent or mixtures thereof. The application of the coatings from non-aqueous solvents may be preferred to limit the exposure of the product to water during processing and maintain anhydrous process conditions. Exemplary materials are disclosed in U.S. Pat. Nos. 4,576,604 and 4,673,405, and the text Pharmaceutical Dosage Forms: Tablets Volume L Second Edition. (A. Lieberman. ed. 1989, Marcel Dekker, Inc.) the relevant disclosures of which are hereby incorporated by reference. In some embodiments, the rapidly dissolving coat will be soluble in saliva, gastric juices, or acidic fluids. Suitable materials include by way of example and without limitation, water soluble polysaccharide gums such as carrageenan, fucoidan, gum ghatti, tragacanth, arabinogalactan, pectin, and xanthan; water-soluble salts of polysaccharide gums such as sodium alginate, sodium tragacanthin, and sodium gum ghattate; water-soluble hydroxyalkylcellulose wherein the alkyl member is straight or branched of 1 to 7 carbons such as hydroxymethylcellulose, hydroxyethylcellulose, and hydroxypropylcellulose; synthetic water-soluble cellulose-based lamina formers such as methyl cellulose and its hydroxyalkyl methylcellulose cellulose derivatives such as a member selected from the group consisting of hydroxyethyl methylcellulose, hydroxypropyl methylcellulose, and hydroxybutyl methylcellulose; other cellulose polymers such as sodium carboxymethylcellulose; and other materials known to those of ordinary skill in the art. Other lamina forming materials that can be used for this purpose include poly(vinylpyrrolidone), polyvinylalcohol, polyethylene oxide, a blend of gelatin and polyvinyl-pyrrolidone, gelatin, glucose, saccharides, povidone, copovidone, poly(vinylpyrrolidone)-poly(vinyl acetate) copolymer. The artisan of ordinary skill will recognize that the above-noted materials include film-forming polymers.

Other materials which can be used in the water soluble coating include hydroxypropylcellulose, microcrystalline cellulose (MCC, Avicel.TM. from FMC Corp.), poly(ethylene-vinyl acetate) (60:40) copolymer (EVAC from Aldrich Chemical Co.), 2-hydroxyethylmethacrylate (HEMA), MMA, terpolymers of HEMA:MMA:MA synthesized in the presence of N,N′-bis(methacryloyloxyethyloxycarbonylamino)-azobenzene, azopolymers, and calcium pectinate can be included in the water soluble coat.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “patient” or “subject” are taken to mean warm blooded animals such as mammals, for example, cats, dogs, mice, guinea pigs, horses, bovine cows, sheep and humans.

A formulation of the invention will comprise a magnesium salt present in an effective amount. By the term “effective amount” is meant the amount or quantity of magnesium salt that is sufficient to elicit the required or desired response, or in other words, the amount that is sufficient to elicit an appreciable biological response when administered to a subject.

When formulated into a dosage form, the composition can be present in a tablet, capsule, pill, troche, stick, granule, pellet, or powder. The preferred embodiments are compressed dosage forms for oral ingestion.

The solid composition of the invention can be used to treat a wide range of magnesium related disorders. For example, one or more unit doses of the solid composition can be used to treat hypomagnesemia, certain cardiac arrhythmias (such as atrial fibrillation, premature atrial and ventricular beats, ventricular tachycardia and ventricular fibrillation), torsade de pointes, and eclampsia. It is also useful as a laxative and antacid. Magnesium may also have value for the prevention of osteoporosis and for the management of migraine headaches in some. The solid composition may help ameliorate premenstrual syndrome, type 2 diabetes mellitus and hypertension.

The magnesium may have anti-osteoporotic activity, anti-arrhythmic activity, activity in the management of preeclampsia, anti-hypertensive activity, glucose-regulatory activity, bronchodilatory activity, myocardial protective activity during an acute myocardial infarction, and anti-migraine activity.

The magnesium may also be effective in treating cardiac arrhythmias in those who are not magnesium deficient. Magnesium sulfate is widely used to prevent eclamptic seizures in pregnant women with hypertension. Magnesium may also protect against damage to the endothelium by reactive oxygen species. It may also act as an anticonvulsant via neuronal calcium-channel blockade and antagonism of the glutamate N-methyl-D-aspartate (NMDA) receptor.

The composition of the invention may help prevent or reduce the incidence of cerebral palsy and mental retardation in early pre-term infants. It may possess significant neuroprotective effects. The magnesium may also be used in protecting against atherosclerosis since magnesium deficiency promotes vascular damage and other atherosclerotic processes. In addition, supplemental magnesium may lower serum cholesterol and triglyceride levels and inhibit atherosclerotic lesions in mammals.

Magnesium daily may significantly improve insulin response and action, compared with placebo. Magnesium may promote bronchodilation and improve lung function in some asthmatic patients especially in patients treated in emergency departments for severe acute asthma. Magnesium may also lower the incidence of airway reactivity and respiratory symptoms.

Alcohol is known to be a potent magnesium diuretic. Supplemental magnesium has shown benefit in some alcoholics. Supplemental magnesium may improve a number of metabolic variables and muscle strength in chronic alcoholics by improving liver cell function and electrolyte status. Magnesium supplementation in combination with phenobarbital therapy may be effective in easing the symptoms of alcohol withdrawal.

Magnesium supplementation may significantly protect post-menopausal women from osteoporosis. The magnesium supplementation may also significantly increased bone density.

The magnesium salt containing composition of the invention may also be used in combination with one or more other agents to enhance the clinical benefit provided by the magnesium salt. For example, concomitant use of non-digestible oligosaccharides and magnesium may increase the colonic absorption of magnesium.

Typical doses of magnesium (expressed as elemental magnesium) range from 100 to 350 milligrams daily. The table below includes dosages recommended by The Food and Nutrition Board of the Institute of Medicine of the United States National Academy of Sciences.

| | |
|---|---|
| Infants | (AI) |
| 0 through 6 months | 30 mg/day |
| 7 through 12 months | 75 mg/day |
| Children | (RDA) |
| 1 through 3 years | 80 mg/day |
| 4 through 8 years | 130 mg/day |
| Boys | |
| 9 through 13 years | 240 mg/day |
| 4 through 18 years | 410 mg/day |
| Girls | |
| 9 through 13 years | 240 mg/day |
| 4 through 18 years | 360 mg/day |
| Men | |
| 19 through 30 years | 400 mg/day |
| 31 through 50 years | 420 mg/day |
| 51 through 70 years | 420 mg/day |
| Greater than 70 years | 420 mg/day |
| Women | |
| 19 through 30 years | 310 mg/day |
| 31 through 50 years | 320 mg/day |
| 51 through 76 years | 320 mg/day |
| Greater than 70 years | 320 mg/day |
| Pregnancy | |
| 14 through 18 years | 400 mg/day |
| 19 through 30 years | 350 mg/day |
| 31 through 50 years | 380 mg/day |
| Lactation | |
| 14 through 18 years | 360 mg/day |
| 19 through 30 years | 310 mg/day |
| 31 through 50 years | 320 mg/day |

“AI” denotes adequate intake.
“RDA” denotes recommended daily allowance.

The Food and Nutrition Board has recommended the following upper limits (UL) for supplementary magnesium (i.e., nonfood source magnesium).

| | |
|---|---|
| Infants | (UL) |
| 0 through 12 months | Not possible to establish for supplementary |
| | magnesium |
| Children | |
| 1 through 3 years | 65 mg of supplementary magnesium |
| 4 through 8 years | 110 mg of supplementary magnesium |
| Pregnancy | |
| 14 through 50 years | 350 mg of supplementary magnesium |
| Lactation | |
| 14 through 50 years | 350 mg of supplementary magnesium |
| Adolescents and | 350 mg of supplementary magnesium |
| Adults | |

It should be noted that the amounts above concern administration of magnesium regardless of the counterion complexed with the magnesium. Therefore, higher molecular weight salts will generally require administration of higher amounts as compared to lower molecular weight salts. The following table described the weight percent of magnesium contained in magnesium salts for varying solubility.

| Magnesium Salt | % (w/w) Mg in Salt |
|---|---|
| MgO | 60.31% |
| Mg(OH)₂ | 41.68% |
| MgF₂ | 39.01% |
| MgCl₂ | 25.53% |
| MgSO₄ | 20.19% |
| Mg Carbonate | 41% |
| Mg Gluconate | 5.89% |
| Mg Lactate | 12.01% |

Specific embodiments of the invention include those wherein the magnesium salt contains at least 39-75% wt. magnesium.

The amount of magnesium salt administered to a subject may be varied as detailed herein or as known by artisans in the art of magnesium supplementation. A single unit of a dosage form containing the composition of the invention may include a therapeutic amount or sub-therapeutic amount of magnesium present as a magnesium salt. Therefore, one or more units of a dosage form may be administered to a subject per day.

In view of the above description and the examples below, one of ordinary skill in the art will be able to practice the invention as claimed without undue experimentation. The foregoing will be better understood with reference to the following examples that detail certain procedures for the preparation of compositions and formulations according to the present invention. All references made to these examples are for the purposes of illustration. The following examples should not be considered exhaustive, but merely illustrative of only a few of the many embodiments contemplated by the present invention.

### EXAMPLE 1

An exemplary compressed tablet according to the invention can be made using the following general procedure, wherein the magnesium salt and filler excipients are charged to a mixing apparatus and blended to obtain a homogenous mixture. Additional fillers or other materials such as disintegrants, glidants, and/or lubricants may be added to the mixer and blending continued. The final blend is then transferred to a suitable molding apparatus such as a tablet press for the preparation of the individual dosage units. This is the process of direct compression. The formed dosage units may then be suitably packaged for storage, distribution, or sale, overencapsulated and packaged, or subsequently processed (i.e. coated) and packaged.

### EXAMPLE 2

An exemplary compressed tablet according to the invention can be made using the following alternate general procedure, wherein the magnesium salt and one or more materials are combined and then agglomerated. Dry granulation is typically an agglomeration method whereby powders are granulated by mechanical compression and milling. Slugging is a dry granulation technique where a blend containing a magnesium salt is compressed into large tablets or “slugs”. The slugs are then milled or ground to produce agglomerates. Roller compaction is a dry granulation technique where a blend containing a magnesium salt is compressed into large flat pieces or ribbons. The flat pieces or ribbons are then milled or ground to produce agglomerates. The agglomerates produced by slugging or roller compaction may then be compressed into tablets or subsequently blended with additional materials then compressed into tablets. The formed dosage units may then be suitably packaged for storage, distribution, or sale, over-encapsulated and packaged, or subsequently processed (i.e. coated) and packaged.

### EXAMPLE 3

Magnesium Oxide Tablet Monograph USP <711> Dissolution.

USP 27/NF22 specification for magnesium oxide tablets is not less than 75% (O) of the labeled amount of MgO is dissolved in 45 minutes. Dissolution testing is performed using apparatus II with 900 mL of 0.1 N hydrochloric acid maintained at 37° C. and agitated at 75 rpm. Samples are withdrawn at after 45 minutes of testing. The amount of MgO dissolved is then determined using atomic absorption (AA) spectrophotometry at a wavelength of 285.2 nm using filtered portions of the solution under test, diluted with Dissolution Medium. A standard curve is generated using a magnesium standard solution of known concentration in the same medium.

USP <701> Disintegration.

An exemplary tablet was placed in each of the six tubes of the basket using water maintained at 37±2° for 30 minutes. The time required was recorded for the first and last tablet to disintegrate. If the tablets did not disintegrate within 30 minutes, the disintegration time was recorded as greater than 30 minutes.

USP <1216> Tablet Friability.

The tablet friability apparatus is rotated at 25±1 rpm for 100 rotations. For tablets with a unit mass equal to or less than 650 mg, a sample of whole tablets corresponding to 6.5 g is used. For tablets with a unit mass of more than 650 mg, a sample of 10 whole tablets is used. The tablets are accurately weighed prior to and after testing and the weight loss is expressed as a percentage. The USP recommendation is a maximum weight loss of not more than 1% is considered acceptable for most products.

Tablet Hardness

The crushing strength, or hardness, of each tablet was measured using a tablet hardness tester such as a VanKel VK200.

### EXAMPLE 4

An exemplary compressed tablet according to the invention can be made using the following general procedure.

MgO tablets were prepared by direct compression to contain 400 mg of MgO per tablet. Tablets were prepared with a 2% overage of magnesium oxide to account for assay reported on the manufacturer's Certificate of Analysis. The manufacturing process (direct compression) entailed sieving and blending the magnesium oxide, polyethylene glycol, poloxamer, crospovidone, and colloidal silicon dioxide in a 5 cubic foot v-shell blender for 300 revolutions. Magnesium stearate was then sieved into the blender, and the powder mass was blended for an additional 60 revolutions. The resulting blend was then tableted on a 45 station BB2 tablet press tooled with 11 mm round, shallow cup concave tooling. The turret speed was adjusted to provide an output of 1400 tablets per minute. The tablets exhibited acceptable weight variation and a hardness range of approximately 6 kp to 8 kp. The friability of the tablets was determined to be about 0.6%, and the tablets exhibited a disintegration time of approximately 9 seconds.

The tablets were made according to the following formula specifications.

Lot No.: 2003-018-46

| Compound | mg/Tablet | g/100,000 Tablets |
|---|---|---|
| MgO, Granular | 408.0 | 40,800 |
| Polyethylene Glycol 8000 | 60.0 | 6,000 |
| Poloxamer 188 | 10.0 | 1,000 |
| Crospovidone | 40.0 | 4,000 |
| Colloidal Silicon Dioxide | 2.6 | 260 |
| Magnesium Stearate | 2.6 | 260 |
| Total | 523.2 | 52,320 |

The bulk tablets were subsequently packaged in high density polyethylene bottles and closed with an aluminum induction seal and a polypropylene child resistant cap. The bottled product was then stored at 40° C. and 75% relative humidity. The influence of storage on the dissolution of these tablets is shown in FIG. 6.

### EXAMPLE 5

An exemplary compressed tablet according to the invention can be made using the following general procedure.

MgO tablets were prepared by direct compression to contain 400 mg of MgO per tablet. Tablets were prepared with a 2% overage of magnesium oxide to account for assay reported on the manufacturer's Certificate of Analysis. The manufacturing process (direct compression) entailed sieving and blending the magnesium oxide, polyethylene glycol, poloxamer, crospovidone, and colloidal silicon dioxide in a 5 cubic foot v-shell blender for 300 revolutions. Magnesium stearate was then sieved into the blender, and the powder mass was blended for an additional 60 revolutions. The resulting blend was then tableted on a 45 station BB2 tablet press tooled with 11 mm round, shallow cup concave tooling. The turret speed was adjusted to provide an output of 1200 tablets per minute. The tablets exhibited acceptable weight variation and a hardness range of approximately 10 kp to 16 kp. The friability of the tablets was determined to be about 0.6%, and the tablets exhibited a disintegration time of approximately 20 seconds.

The tablets were made according to the following formula specifications.

Lot No.: 2003-018-47

| Compound | mg/Tablet | g/100,000 Tablets |
|---|---|---|
| MgO, Granular | 408.0 | 40,800 |
| Polyethylene Glycol 8000 | 80.0 | 8,000 |
| Poloxamer 188 | 10.0 | 1,000 |
| Crospovidone | 50.0 | 5,000 |
| Colloidal Silicon Dioxide | 2.7 | 270 |
| Magnesium Stearate | 2.7 | 270 |
| Total | 553.4 | 55,340 |

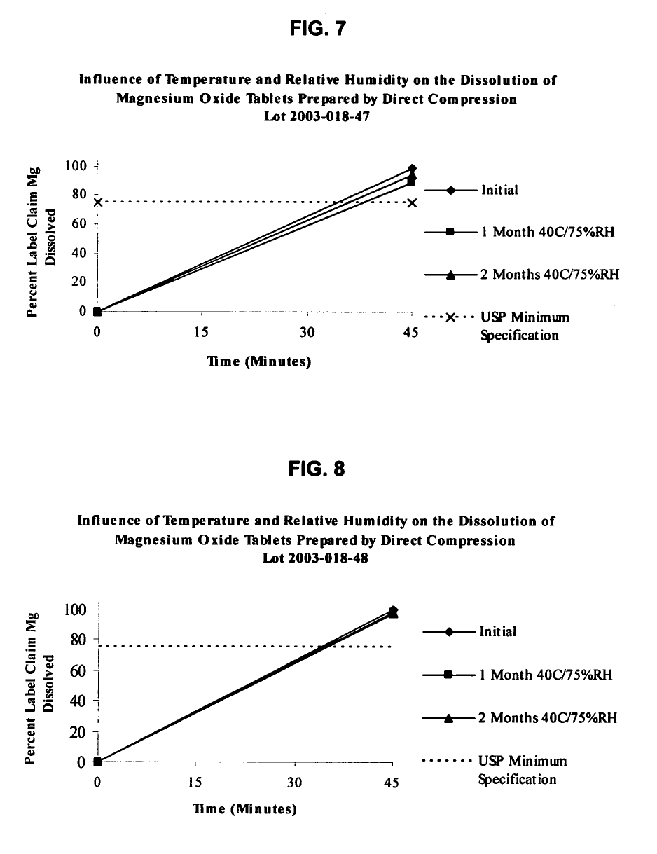

The bulk tablets were subsequently packaged in high density polyethylene bottles and closed with an aluminum induction seal and a polypropylene child resistant cap. The bottled product was then stored at 40° C. and 75% relative humidity. The influence of storage on the dissolution of these tablets is shown in FIG. 7.

### EXAMPLE 6

An exemplary compressed tablet according to the invention can be made using the following general procedure.

MgO tablets were prepared by direct compression to contain 400 mg of MgO per tablet. Tablets were prepared with a 2% overage of magnesium oxide to account for assay reported on the manufacturer's Certificate of Analysis. The manufacturing process (direct compression) entailed sieving and blending the magnesium oxide, polyethylene glycol, poloxamer, crospovidone, and colloidal silicon dioxide in a 5 cubic foot v-shell blender for 300 revolutions. Magnesium stearate was then sieved into the blender, and the powder mass was blended for an additional 60 revolutions. The resulting blend was then tableted on a 45 station BB2 tablet press tooled with 11 mm round, shallow cup concave tooling. The turret speed was adjusted to provide an output of 1200 tablets per minute. The tablets exhibited acceptable weight variation and a hardness range of approximately 10 kp to 13 kp. The friability of the tablets was determined to be about 0.5%, and the tablets exhibited a disintegration time of approximately 20 seconds.

The tablets were made according to the following formula specifications.

Lot No.: 2003-018-48

| Compound | mg/Tablet | g/100,000 Tablets |
|---|---|---|
| MgO, Granular | 408.0 | 40,800 |
| Polyethylene Glycol 8000 | 100.0 | 10,000 |
| Poloxamer 188 | 10.0 | 1,000 |
| Crospovidone | 62.5 | 6,250 |
| Colloidal Silicon Dioxide | 2.9 | 290 |
| Magnesium Stearate | 2.9 | 290 |
| Total | 586.3 | 58,630 |

The bulk tablets were subsequently packaged in high density polyethylene bottles and closed with an aluminum induction seal and a polypropylene child resistant cap. The bottled product was then stored at 40° C. and 75% relative humidity. The influence of storage on the dissolution of these tablets is shown in FIG. 8.

### EXAMPLE 7

An exemplary compressed tablet according to the invention can be made using the following general procedure.

MgO tablets were prepared by direct compression to contain 400 mg of MgO per tablet. Tablets were prepared with a 2% overage of magnesium oxide to account for assay reported on the manufacturer's Certificate of Analysis. The manufacturing process (direct compression) entailed sieving and blending the magnesium oxide, polyethylene glycol, poloxamer, ethylcellulose, crospovidone, and colloidal silicon dioxide in a 5 cubic foot v-shell blender for 300 revolutions. Magnesium stearate was then sieved into the blender, and the powder mass was blended for an additional 60 revolutions. The resulting blend was then tableted on a 45 station BB2 tablet press tooled with 11 mm round, shallow cup concave tooling. The turret speed was adjusted to provide an output of 1400 tablets per minute. The tablets exhibited acceptable weight variation and a hardness range of approximately 21 kp to 25 kp. The friability of the tablets was determined to be about 0.1%, and the tablets exhibited a disintegration time of approximately 40 seconds.

The tablets were made according to the following formula specifications.

Lot No.: 2003-018-49

| Compound | mg/Tablet | g/100,000 Tablets |
|---|---|---|
| MgO, Granular | 408.0 | 40,800 |
| Polyethylene Glycol 8000 | 80.0 | 8,000 |
| Poloxamer 188 | 10.0 | 1,000 |
| Ethylcellulose | 28.7 | 2,870 |
| Crospovidone | 50.0 | 5,000 |
| Colloidal Silicon Dioxide | 2.9 | 290 |
| Magnesium Stearate | 2.9 | 290 |
| Total | 582.5 | 58,250 |

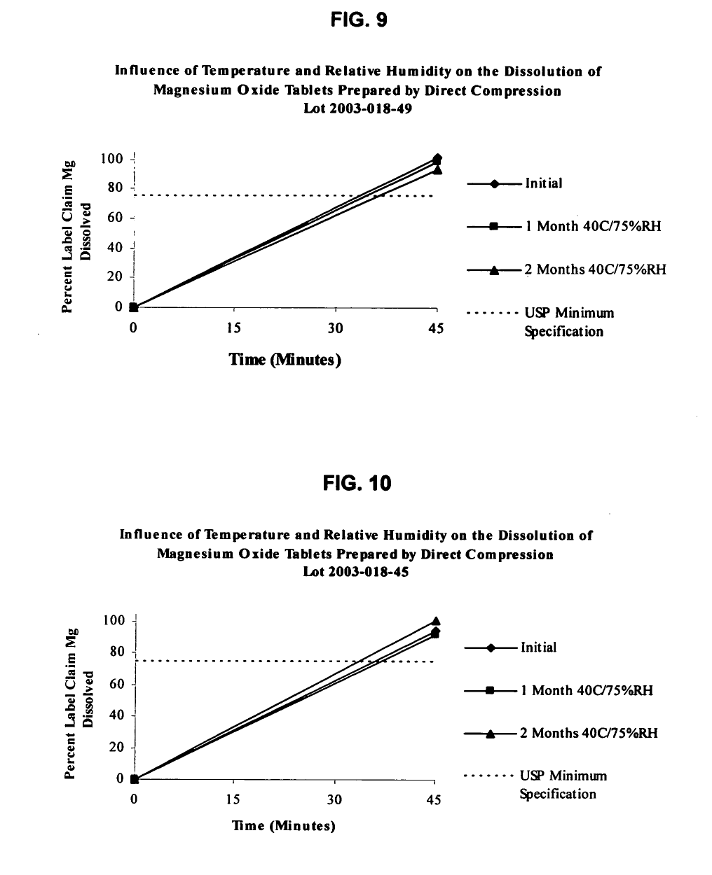

The bulk tablets were subsequently packaged in high density polyethylene bottles and closed with an aluminum induction seal and a polypropylene child resistant cap. The bottled product was then stored at 40° C. and 75% relative humidity. The influence of storage on the dissolution of these tablets is shown in FIG. 9.

### EXAMPLE 8

An exemplary compressed tablet according to the invention can be made using the following general procedure.

MgO tablets were prepared by direct compression to contain 400 mg of MgO per tablet. Tablets were prepared with a 2% overage of magnesium oxide to account for assay reported on the manufacturer's Certificate of Analysis. The manufacturing process (direct compression) entailed sieving and blending the magnesium oxide, lactose, polyethylene glycol, poloxamer, crospovidone, ethylcellulose, and colloidal silicon dioxide in a 5 cubic foot v-shell blender for 300 revolutions. Magnesium stearate was then sieved into the blender, and the powder mass was blended for an additional 60 revolutions. The resulting blend was then tableted on a 16 station RB2 tablet press tooled with embossed modified oval tooling. The turret speed was adjusted to 30 revolutions per minute. The tablets exhibited acceptable weight variation and a hardness range of approximately 12 kp to 17 kp. The friability of the tablets was determined to be about 0.1%, and the tablets exhibited a disintegration time of approximately 30 seconds.

The tablets were made according to the following formula specifications.

Lot No.: 2003-018-45

| Compound | mg/Tablet | g/100,000 Tablets |
|---|---|---|
| MgO, Granular | 408.0 | 40,800 |
| Lactose, Anhydrous | 85.5 | 8,550 |
| Polyethylene Glycol 8000 | 100.0 | 10,000 |
| Poloxamer 188 | 10.0 | 1,000 |
| Ethylcellulose | 35.0 | 3,500 |
| Crospovidone | 62.5 | 6,250 |
| Colloidal Silicon Dioxide | 3.5 | 350 |
| Magnesium Stearate | 3.5 | 350 |
| Total | 708.0 | 70,800 |

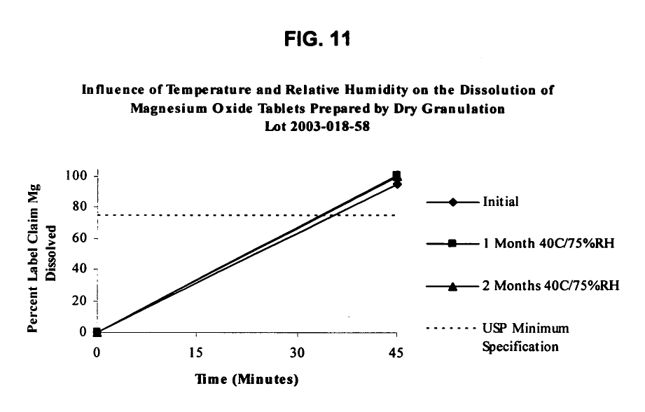

The bulk tablets were subsequently packaged in high density polyethylene bottles and closed with an aluminum induction seal and a polypropylene child resistant cap. The bottled product was then stored at 40° C. and 75% relative humidity. The influence of storage on the dissolution of these tablets is shown in FIG. 10.

### EXAMPLE 9

An exemplary compressed tablet according to the invention can be made using the following general procedure.

MgO tablets were prepared by dry granulation to contain 400 mg of MgO per tablet. Tablets were prepared with a 2% overage of magnesium oxide to account for assay reported on the manufacturer's Certificate of Analysis. The tablets were prepared by blending and compressing the components identified below (in italics) into slugs. The slugs were then milled before combining with ethylcellulose, crospovidone, and colloidal silicon dioxide. The granulation and extragranular excipients were blended for 300 revolutions before the addition of magnesium stearate. The final blend was mixed for an additional 60 revolutions. The final blend was then tableted on a 45 station BB2 tablet press tooled with 11 mm round, shallow cup concave tooling. The turret speed was adjusted to provide an output of 1400 tablets per minute.

For tablets compressed at the target weight, tablets exhibited a hardness range of approximately 15 to 20 kp. The friability of the tablets was determined to be about 0.2%, and the tablets exhibited a disintegration time of approximately 90 seconds.

The tablets were made according to the following formula specifications.

−Lot No.: 2003-018-58

| Compound | mg/Tablet | g/100,000 Tablets |
|---|---|---|
| MgO, Granular | 408.00 | 40,800 |
| Polyethylene Glycol 8000 | 100.00 | 10,000 |
| Poloxamer 188 | 10.00 | 1,000 |
| Magnesium Stearate | 2.54 | 254 |
| Ethylcellulose | 30.60 | 3,060 |
| Crospovidone | 62.50 | 6,250 |
| Colloidal Silicon Dioxide | 3.00 | 300 |
| Magnesium Stearate | 3.00 | 300 |
| Total | 619.64 | 61,964 |

The bulk tablets were subsequently packaged in high density polyethylene bottles and closed with an aluminum induction seal and a polypropylene child resistant cap. The bottled product was then stored at 40° C. and 75% relative humidity. The influence of storage on the dissolution of these tablets is shown in FIG. 11.

### EXAMPLE 10

The following methods were used to package the present formulation in order to conduct side-by-side comparative analyses of storage stability under a predetermined set of conditions to the commercial prior art formulations.

The initial dissolution profile for each of the prior art and present formulations is determined as described herein. Prior art formulations are obtained in their original packaging configurations. Formulations of the invention are each placed in respective sealed containers. Sealing of the container-enclosure system is carried out by tightening the closure onto the bottle using the manufacturer's recommended applied torque and induction sealing the aluminum seal into place. After induction sealing, the enclosure is again tightened to the manufacturer's recommended range. U.S.P. <671> provides recommendations for the applied torque of various diameter enclosures. The unopened prior art and sealed present formulations are then exposed to 40° C. and 75% RH. At monthly intervals, the dissolution profile of the formulations is determined again. The results are plotted in the attached figures.

The current invention was evaluated in HDPE containers from Alcan Packaging (Millville, N.J.) and Quality Container (Ypsilanti, Mich.) with CRC closures from Rexam (Evansville, Ind.). Other suppliers of similar containers and closures are known to those of ordinary skill in the arts.

The disclosures of the references cited herein are hereby incorporated in their entirety.

The above is a detailed description of particular embodiments of the invention. It will be appreciated that, although specific embodiments of the invention have been described herein for purposes of illustration, various modifications may be made without departing from the spirit and scope of the invention. Accordingly, the invention is not limited except as by the appended claims. All of the embodiments disclosed and claimed herein can be made and executed without undue experimentation in light of the present disclosure.

1. A rapidly dissolving solid oral compressed composition comprising:
a. one or more magnesium salts;
b. one or more hydrophilic polymers;
c. one or more disintegrants;
d. optionally one or more surfactants;
e. optionally one or more glidants;
f. optionally one or more fillers; and
g. optionally one or more lubricants;
h. wherein the composition provides a substantially stable dissolution profile when evaluated in vitro according to USP <711> for the one or more magnesium salts when the composition is stored for at least two months at 40° C. and 75% relative humidity in a sealed container-enclosure system.

2. The composition of claim 1, wherein the magnesium salt is MgO, Mg Carbonate, MgF₂, or Mg(OH)₂.

3. The composition of claim 1, wherein the one or more hydrophilic polymers is a combination of polymers.

4. The composition of claim 3, wherein the one or more hydrophilic polymers is selected from the group consisting of polyethylene glycol, poloxamer, povidone, and co-povidone.

5. The composition of claim 1, wherein the disintegrant is selected from the group consisting of: crospovidone, low substituted hydroxypropylcellulose, croscarmellose sodium, and sodium starch glycolate.

6. The composition of claim 1 further comprising a coating surrounding the compressed composition.

7. The composition of claim 1, wherein the composition is included in a tablet or capsule dosage form

8. The composition of claim 1, wherein the composition is prepared by dry granulation.

9. The composition of claim 1, wherein the composition is prepared by direct compression.

10. The composition of claim 1, wherein the magnesium salt is a sparingly soluble, slightly soluble, very slightly soluble, practically insoluble or insoluble salt.

11. The composition of claim 1, wherein the magnesium salt is the only component present in a therapeutically effective amount.

12. The composition of claim 1 further comprising a capsule shell within which the compressed composition is enclosed.

13. The composition of claim 1, wherein the compressed composition is tablet or pill.

14. The composition of claim 13, wherein the tablet or pill exhibits a hardness of about 4 kp to about 50 kp.

15. The composition of claim 1, wherein the dissolution medium for evaluation is dilute hydrochloric acid.

16. The composition of claim 1, wherein the solid oral compressed composition is in a sealed container-enclosure system during storage.

17. The composition of claim 16, wherein
a. the container comprises a material selected from the group consisting of glass, metal, or polymers;
b. the enclosure comprises a material selected from the group consisting of metal or polymers; and
c. the container-enclosure system is sealed by mechanical tightening and induction sealing of a taper evident liner onto the orifice of the container.

18. The composition of claim 17, wherein
a. the container comprises high density polyethylene;
b. the enclosure comprises CRC or non-CRC polypropylene; and
c. the container-enclosure system is sealed using an appropriate torque and an induction sealed aluminum tamper evident liner.

19. The composition of claim 18, wherein the compressed composition is prepared by direct compression.

20. The composition of claim 1, where in the compressed composition contains less than 7.5% water.

21. The composition of claim 20, wherein the compressed composition contains less than 5.5% water.

22. The composition of claim 21, wherein the compressed composition contains less than 4% water.

23. A solid oral dosage form comprising:
a. a compressed composition comprising:
i. one or more magnesium salts;
ii. one or more hydrophilic polymers;
iii. one or more disintegrants;
iv. optionally one or more surfactants;
v. optionally one or more glidants;
vi. optionally one or more fillers; and
vii. optionally one or more lubricants; wherein
viii. the composition provides a substantially stable dissolution profile when evaluated in vitro according to USP <711> for the one or more magnesium salts when the composition is stored for at least two months at 40° C. and 75% relative humidity in a sealed container-enclosure system.

24. The dosage form of claim 23, wherein the magnesium salt is the only component present in a therapeutically effective amount.

25. The dosage form of claim 23, wherein the magnesium salt is a sparingly soluble, slightly soluble, very slightly soluble, practically insoluble or insoluble salt.

26. The dosage form of claim 25, wherein the magnesium salt is selected from the group consisting of MgO, Mg(OH)₂, MgF₂, and Mg Carbonate.

27. The dosage form of claim 25, wherein the one or more hydrophilic polymers is a combination of polymers.

28. The dosage form of claim 23, wherein the one or more hydrophilic polymers is a combination of polymers.

29. The dosage form of claim 23, wherein the composition is prepared by dry granulation.

30. The dosage form of claim 23, wherein the composition is prepared by direct compression.

31. The dosage form of claim 23, wherein the composition contains less than 7.5% water.

32. The dosage form of claim 31, wherein the composition is prepared by dry granulation.

33. The dosage form of claim 31, wherein the composition is prepared by direct compression.

34. The dosage form of claim 23 further comprising a coating surrounding the compressed composition.

35. The dosage form of claim 23 further comprising a capsule shell within which the compressed composition is enclosed.

36. A compressed composition adapted for oral administration to a subject comprising:
a. one or more magnesium salts;
b. one or more hydrophilic polymers;
c. one or more disintegrants;
d. optionally one or more surfactants;
e. optionally one or more glidants;
f. optionally one or more fillers; and
g. optionally one or more lubricants; wherein
h. the magnesium salt is the only component present in a therapeutically effective amount;
i. the composition provides a substantially stable dissolution profile when evaluated in vitro according to USP <711> for the one or more magnesium salts when the composition is stored for at least two months at 40° C. and 75% relative humidity in a sealed container-enclosure system; and
j. the composition contains less than 7.5% water.

37. The composition of claim 36, wherein the magnesium salt is a sparingly soluble, slightly soluble, very slightly soluble, practically insoluble or insoluble salt.

38. The composition of claim 37, wherein the magnesium salt is selected from the group consisting of MgO, Mg(OH)₂, MgF₂, and Mg Carbonate.

39. The composition of claim 37, wherein the composition is prepared by direct compression or dry granulation.

40. The composition of claim 36, wherein the composition is prepared by direct compression or dry granulation.

41. The composition of claim 36, wherein the composition is prepared by a process that does not include the addition of water.

42. The composition of claim 36, wherein
a. the container comprises a material selected from the group consisting of glass, metal, or polymers;
b. the enclosure comprises a material selected from the group consisting of metal or polymers; and
c. the container-enclosure system is sealed by mechanical tightening and induction sealing of a taper evident liner onto the orifice of the container.

43. The composition of claim 36, wherein the composition excludes microcrystalline cellulose.

44. A rapidly dissolving solid oral compressed composition comprising:
a. one or more magnesium salts;
b. one or more hydrophilic polymers;
c. one or more disintegrants; and
d. at least one or more of the following: surfactant, glidant, filler, and lubricant; wherein
e. the composition provides a substantially stable dissolution profile when evaluated in vitro according to USP <711> for the one or more magnesium salts when the composition is stored for at least two months at 40° C. and 75% relative humidity in a sealed container-enclosure system;
f. the composition is prepared by a substantially anhydrous process; and
g. the magnesium salt is a sparingly soluble, slightly soluble, very slightly soluble, practically insoluble or insoluble salt.

45. The composition of claim 44, wherein the magnesium salt is selected from the group consisting of MgO, Mg(OH)₂, MgF₂, and Mg Carbonate.

46. The composition of claim 44, wherein the composition is prepared by direct compression or dry granulation.

47. The composition of claim 44, wherein the composition contains less than 7.5% water.

48. The dosage form of claim 44, wherein the one or more hydrophilic polymers is a combination of polymers.

49. The composition of claim 44, wherein the magnesium salt is the only component present in a therapeutically effective amount.

50. The composition of claim 44, wherein the one or more hydrophilic polymers is selected from the group consisting of polyethylene glycol, poloxamer, povidone, and co-povidone.

51. The composition of claim 44, wherein the disintegrant is selected from the group consisting of: crospovidone, low substituted hydroxypropylcellulose, croscarmellose sodium, and sodium starch glycolate.

52. A rapidly dissolving solid oral compressed composition comprising:
a. one or more magnesium salts selected from the group consisting of MgO, Mg(OH)₂, MgF₂, and Mg Carbonate;
b. one or more hydrophilic polymers;
c. one or more disintegrants; and
d. at least one or more of the following: surfactant, glidant, filler, and lubricant; wherein
e. the composition provides a substantially stable dissolution profile when evaluated in vitro according to USP <711> for the one or more magnesium salts when the composition is stored for at least two months at 40° C. and 75% relative humidity in a sealed container-enclosure system;
f. the composition is prepared by a substantially anhydrous process;
g. the magnesium salt is a sparingly soluble, slightly soluble, very slightly soluble, practically insoluble or insoluble salt; and
h. the magnesium salt is the only component present in a therapeutically effective amount.

53. The composition of claim 52, wherein the composition is prepared by direct compression or dry granulation.

54. The composition of claim 52, wherein the composition contains less than 7.5% water.

55. The dosage form of claim 52, wherein the one or more hydrophilic polymers is a combination of polymers.

56. The composition of claim 55, wherein the one or more hydrophilic polymers is selected from the group consisting of polyethylene glycol, poloxamer, povidone, and co-povidone.

57. The composition of claim 52, wherein the disintegrant is selected from the group consisting of: crospovidone, low substituted hydroxypropylcellulose, croscarmellose sodium, and sodium starch glycolate.

58. The composition of claim 52, wherein the composition excludes microcrystalline cellulose.
